(19) United States
(12) Patent Application Publication
(10) Pub. No.: US 2009/0013468 A1
(43) Pub. Date: Jan. 15, 2009

(54) Radiotherapy apparatus

(75) Inventors: Duncan Neil Bourne, Redhill (GB); Ralph Streamer, Horsham (GB); Alan Hitchings, Crawley (GB)

Correspondence Address: LAHIVE & COCKFIELD, LLP;FLOOR 30, SUITE 3000, ONE POST OFFICE SQUARE, BOSTON, MA, 02109, US

(73) Assignee: Elekta AB (publ), Stockholm (SE)

(21) Appl. No.: 11/827,320

(22) Filed: Jul. 11, 2007

(51) Int. Cl. A61N 5/00

(52) U.S. Cl. 5/600

(57) ABSTRACT

A patient support system comprises a base, an upstanding support, and a couch attached to the support and having a cantilever section extendable beyond the support; the support including a couch drive means to adjust the position of the couch relative to the support; further comprising a support drive means for translationally driving the support relative to the base in the direction of the cantilever section. This removes inaccuracies from patient position measurements that result from changes in the cantilever geometry during movement of the patient from a measuring position to a treatment position. The support drive means can be located in the base or in the support, and allows the patient support system as a whole to be translated, thereby permitting the patient to be moved into and out of an enclosed treatment area without affecting the vertical location of the patient. The only change to the patient position is in the translational position, which can be calibrated by way of fixed end stops for the support drive means. The present invention further relates to a radiotherapy apparatus, comprising a patient support as set out above, and an enclosed treatment area positioned in line with the cantilever section.

### FIELD OF THE INVENTION

The present invention relates to apparatus for use in radiotherapy.

### BACKGROUND ART

Radiotherapy operates by directing a beam of harmful radiation towards a cancerous region of a patient. Care is needed both to ensure that the prescribed dose is applied to the tumour and also that a minimum dose is applied to healthy tissue. Inefficiencies in either respect reduce the effectiveness of the treatment; if the prescribed dose is not achieved then the tumour will be more likely to recur, whereas excessive dose outside the tumour will damage healthy tissue, may cause side effects, and may slow the patient's recovery from treatment thereby limiting the rate at which doses can be applied.

Care is therefore taken to shape or modulate the beam so as to limit dose rates outside the tumour and maximise dose rates within the tumour. This clearly requires accurate knowledge of the location and extent of the tumour, which is usually obtained via imaging apparatus shortly before the treatment is applied. This data then needs to be correlated with the position of the patient in the radiotherapy apparatus so that an accurate location for the tumour relative to the apparatus can be determined.

Efforts are therefore made to ascertain the patient position. Video and/or laser apparatus can be used, in conjunction with position markers or known features on the patient.

During treatment, patients are placed on a patient support. These are, in effect, servo-controlled couches on which the patient can be placed and which can be moved in up to six degrees of freedom. That movement is used to position the patient accurately, and to ease access to the couch.

The couch is usually supported and controlled from one end, to allow free access beneath the couch for use with rotating-arm or C-arm apparatus, and to allow the couch to be inserted into an enclosed apparatus.

### SUMMARY OF THE INVENTION

The application and maintenance of markers on a patient requires that medical staff have access to and around the patient. Equally, the use of video or laser systems requires a clear line of sight to the patient.

This requirement is incompatible with enclosed radiotherapy systems, in which the patient is inserted into a cylindrical aperture in the apparatus. Accordingly, the patient support that is provided in combination with such apparatus is generally provided with a relatively large range of motion in the horizontal axis parallel to the cylindrical aperture. This allows the patient to be placed on the couch, positioned accurately (or their position determined accurately), and then transported into the cylindrical aperture by translational movement of the couch. Knowledge of the distance moved by the couch should allow the patient's position inside the treatment apparatus to be inferred.

This movement does however introduce an inaccuracy into the patient position. Given that the couch is supported from one end, the translation of the couch will mean that the degree by which the material of the couch flexes under the weight of the patient will change as the couch is translated. That will result in a slight downward movement of the patient. This downward movement will vary with the weight distribution of the patient and will therefore be very difficult to model, predict, and allow for.

The present invention therefore provides a patient support system, comprising a base, an upstanding support, and a couch attached to the support and having a cantilever section extendable beyond the support; the support including a couch drive means to adjust the position of the couch relative to the support; further comprising a support drive means for translationally driving the support relative to the base in the direction of the cantilever section.

The support drive means can be located in the base or in the support, and allows the patient support system as a whole to be translated, thereby permitting the patient to be moved into and out of an enclosed treatment area without affecting the vertical location of the patient. The cantilever section on which the patient is supported maintains the same geometric shape at all times and therefore the positional calibration taken with the patient outside the treatment area remains valid. The only change to the patient position is in the translational position, which can be calibrated by way of fixed end stops for the support drive means.

The support drive means can also drive the support in rotation about a vertical axis. Such rotation is sometimes called for in order to allow the patient to be treated in a plane that excludes radiation-sensitive organs.

The couch drive means can allow one or more of adjustment of the height the couch relative to the base, displacement of the couch in the direction of the cantilever section, displacement of the couch in a direction transverse to the direction of the cantilever section, rotation of the couch about a vertical axis, and rotation of the couch about one or more horizontal axes.

The base can be (simply) a section of floor, but there will usually be some form or structure between the support drive means and the floor, even if only a fixing for attaching the unit to a floor.

The present invention further relates to a radiotherapy apparatus, comprising a patient support as set out above, and an enclosed treatment area positioned in line with the cantilever section.

Optical position detection apparatus are preferably provided, for locating a patient on the couch. Such optical position detection apparatus can comprise a laser alignment system and/or a video camera. Generally, the patient will not be visible to the optical position detection apparatus when the cantilever section is extended into the enclosed treatment area, so the present invention can be employed to obtain a more accurate positional determination in respect of the patient.

### BRIEF DESCRIPTION OF THE DRAWINGS

An embodiment of the present invention will now be described by way of example, with reference to the accompanying figures in which;

FIGS. 1 and 2 show different positions of a known patient support system for use in placing a patient within an enclosed radiotherapy apparatus;

FIGS. 3 and 4 show different positions of a patient support system according to the present invention;

FIG. 5 shows a generic patient support system;

FIG. 6 shows the patient support system of FIG. 5 adapted according to the present invention;

FIG. 7 shows a patient support system according to the present invention prior to insertion into an enclosed radiotherapy apparatus;

FIG. 8 shows the patient support system of FIG. 6 after insertion into an enclosed radiotherapy apparatus; and

FIG. 9 shows an alternative form of patient support system according to the present invention.

### DETAILED DESCRIPTION OF THE EMBODIMENTS

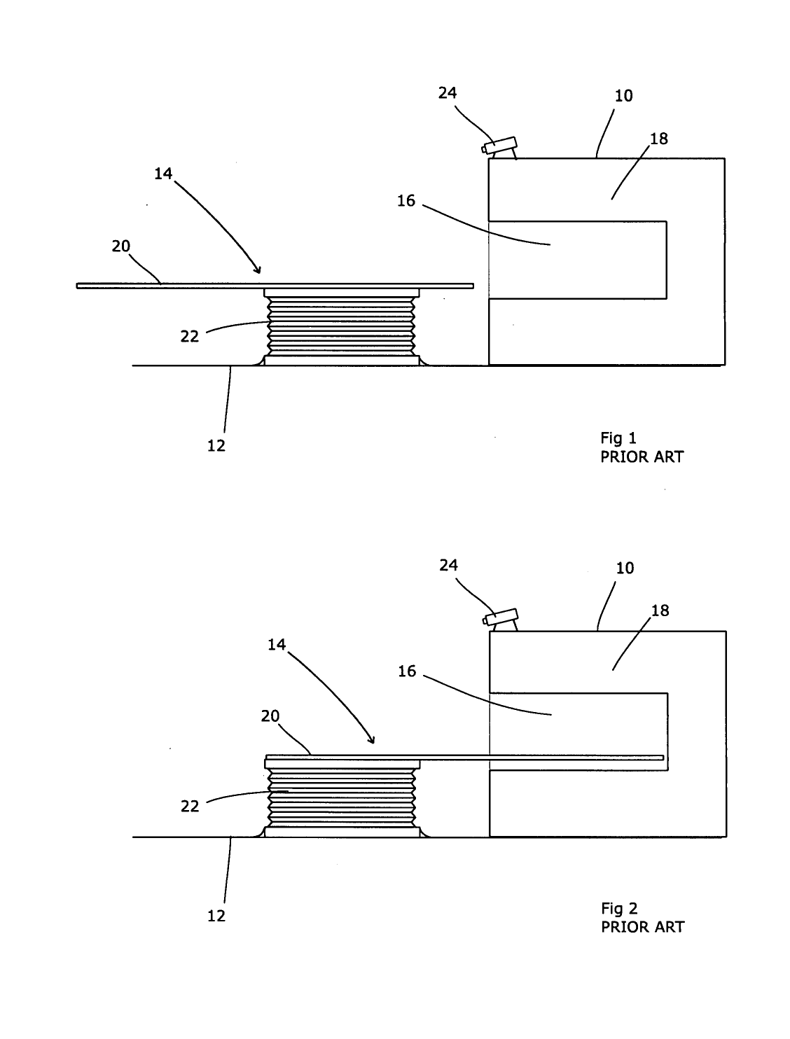

Referring to FIG. 1, a radiotherapeutic apparatus 10 rests on a floor 12. A patient support 14 is placed in front of the radiotherapy apparatus 10 in order to support a patient during treatment. The radiotherapeutic apparatus 10 is generally of the sort in which a cylindrical hole 16 is provided into which the patient is placed for treatment. A generally annular structure 18 is disposed around the cylindrical aperture 16 and contains the necessary radiation sources etc. which are typically able to move around the cylindrical aperture 16 in order to direction radiation towards the patient from a range of directions. By doing so, the dosage applied to areas around (but not within) the target volume can be minimised whilst maximising the dose delivered to the target volume itself.

The patient support 14 consists of a generally horizontal couch 20 onto which the patient is placed. This is held in a desired position by an upright support 22 which is servo-controlled so as to lift and raise the couch 20 as desired. An example of such a support arrangement is shown in WO97/42876, and shows an arrangement by which the couch 20 can be raised and lowered as desired. For example, the couch can be lowered to allow a patient to take up position thereon more easily, and then raised to a suitable height for insertion into the aperture 16.

In addition, modern couches contain the ability to conduct fine adjustment of the couch 20 in all six possible degrees of freedom so as to ensure that the patient is positioned accurately. Modern radiotherapy apparatus, in turn, is able to collimate the beam to an accuracy measured in millimetres at the isocentre or target volume location, and this calls for similarly accurate positioning of the patient. Thus, it is common for the patient position to be measured before the treatment so that the patient is accurately positioned relative to the rotation axis of the device, and (in this embodiment) a video camera 24 is provided for this purpose.

This typically detects the position of markers placed on or in the patient's tissue at known locations, which can therefore be used to calibrate the precise position of the patient. The couch position 20 can then be subjected to fine adjustment so as to bring the target volume in the patient to the required location relative to the radio therapeutic apparatus 10. Other patient location detection systems include laser alignment with suitable markers or structures within the patient, and the like.

Clearly, the video camera 24 cannot see into the aperture 16. As shown, it is positioned on the radio therapeutic apparatus 10, although it will often be located at a different (known) position within the room. In any case, due to the cylindrical nature of the aperture 16, the patient is not usually visible to such positioning apparatus during treatment and therefore patient positioning must be carried out with the patient outside the radio therapeutic apparatus 10.

Once their location has been measured, the patient is moved into the cylindrical aperture 16 as shown in FIG. 2. This is done by suitable motorised movement of the couch 20 relative to the upright support 22, and enables the patient to be placed within the radiotherapeutic apparatus 10 for treatment.

The problem that we have identified with this procedure is that during the initial positioning step shown in FIG. 1, the patient is positioned either directly over the upright support 22, or only projects a short distance off the support 22. After the couch 20 is moved so that the patient is within the cylindrical aperture 16, the patient is then at the end of a long cantilever arm, supported only at one end near (typically) the feet of the patient. Whilst steps can be taken to stiffen the material of the couch 20, such as by suitable material selection and careful design of the structural members making up the couch 20, it will inevitably be the case that there will be some bending of the couch 20 resulting in the patient being slightly lower when within the radio therapeutic apparatus 10 than when being positioned.

This slight sag in the couch 20 will depend on the weight of the patient, the distribution of the patient's weight, the patient's position along the couch 20, and their position within the radiotherapeutic apparatus 10. It will therefore, generally, be very difficult to predict the degree of sag in advance and allow for it. Accordingly, this problem introduces a variably unpredictable error into the patient positioning that will have an adverse affect on treatment.

Generally, inaccuracies in positioning and/or collimation of the beam mean that a greater margin has to be allowed around the tumour in order to ensure complete coverage of the cancerous tissue. This corresponds to a greater irradiation of healthy tissue, and correspondingly collateral damage to healthy parts of the patient. This then results in greater side effects for the patient.

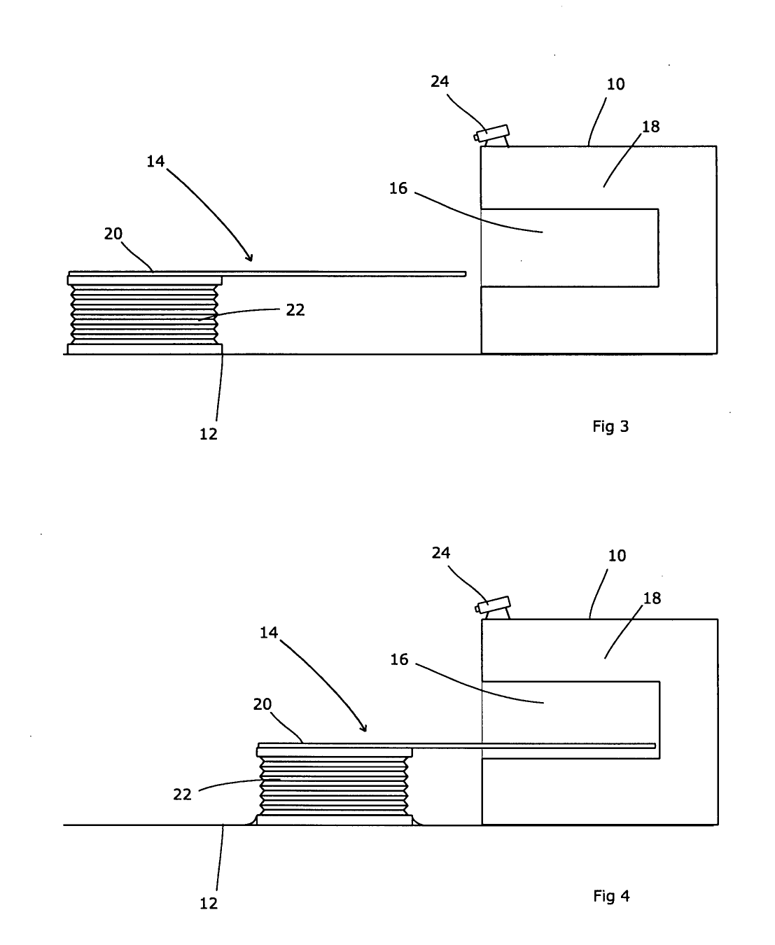

Accordingly, FIGS. 3 and 4 show a patient support according to the present invention. Generally, the arrangement is the same as that shown in FIGS. 1 and 2, and therefore like reference numerals are employed. The principal difference that the upright support 22 is mounted on a moveable base and is able to move along the floor thereby to move the couch 20 into and out of the cylindrical aperture 16. The base can be servo-controlled although it need not be and could be an open loop, end-to-end drive without position feedback.

This means that the patient can be placed onto the couch 20 in the position shown in FIG. 3 and the necessary positioning carried out. Then, instead of translating the couch 20 relative to the upright support 22, the support itself is translated along the floor 12 to the position shown in FIG. 4 so that the patient is now within the cylindrical aperture 16 ready for radiotherapy treatment. However, the patient position on the couch structure 14 now has an identical to that which existed during positioning. The result of this is that the vertical displacement of the patient will not change between positioning and therapy, and the measured position of the patient can be treated with greater confidence.

End stops can be provided on or in the floor 12 to calibrate the movement of the apparatus support 22. It will generally be preferred that the positioning state shown in FIG. 3 will involve the patient support 22 (or a part thereof) being abutted against one end stop and the positioning state shown in FIG. 4 will involve it being positioned against the other end stop—this may be the same or a different part of the support. This provides accurate calibration of the two positions; this could be augmented or replaced by suitable visible markers on the couch 20 within the field of view of the camera 24 or other position-detecting apparatus so that correct movement of the patient could be confirmed.

The apparatus support 22 could be mounted onto runners inset into the floor 12, with the necessary drive means interposed between those runners and the remainder of the apparatus support 22. Alternatively, other arrangements could be adopted as will be apparent to the skilled reader.

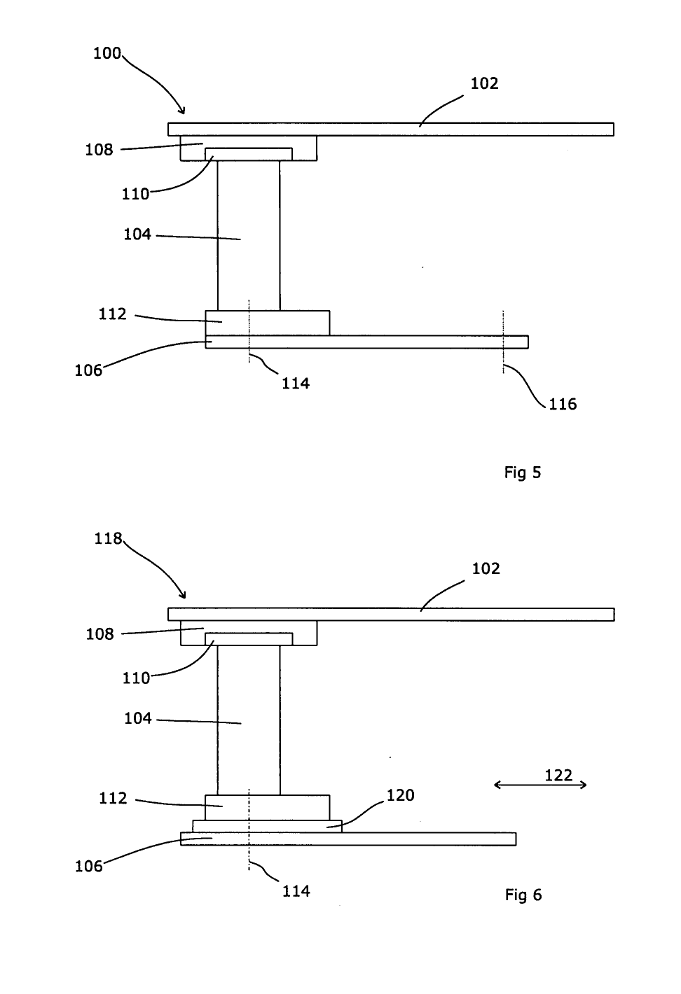

FIG. 5 shows a traditional patient support system 100 in somewhat more detail. The elongate table top 102 is supported by a column 104 which is in turn supported on a base 106. Between the column 104 and the table top 102 are two translational motors 108, 110, one motor 108 acting parallel to the length of the table top 102 and therefore providing longitudinal movement of the table top 102, and one motor 110 acting transverse thereto and therefore providing lateral movement of the table top 102.

A suitable rotational joint is provided at 112 to permit rotation of the column about its own axis 114. Finally, the base is driven in rotation relative to the floor on which it is supported, by way of a base drive motor integrated into the base 106, and adapted to cause rotation thereof about a vertical axis 116 passing through the isocentre of the apparatus.

FIG. 6 shows a patient support 118 according to the present invention. Generally, the design of this patient support 118 is similar to that of the patient support 100 of FIG. 5 and therefore like parts thereof have been given like reference numerals. Instead of a base drive motor that acts in rotation, however, this patient support 118 is provide with a translational base drive motor 120 which is adapted to transport the column 104 and table top 102 bodily along an axis 122 generally in line with the length of the table top 102. Other forms of linear propulsion could of course be employed, such as a pneumatic or hydraulic cylinder.

This allows the table top to slide in and/or out of a bore of an enclosed radiotherapy apparatus (such as an MRI, CT, Polo or Donut style machine) with minimal deflection between the ‘set-up’ and ‘treat’ positions. Generally, the motor can be provided by way of one or more longitudinal rails or guides in the base 106 on which the motor can move under the control of a suitable drive means within the base or the motor section 120.

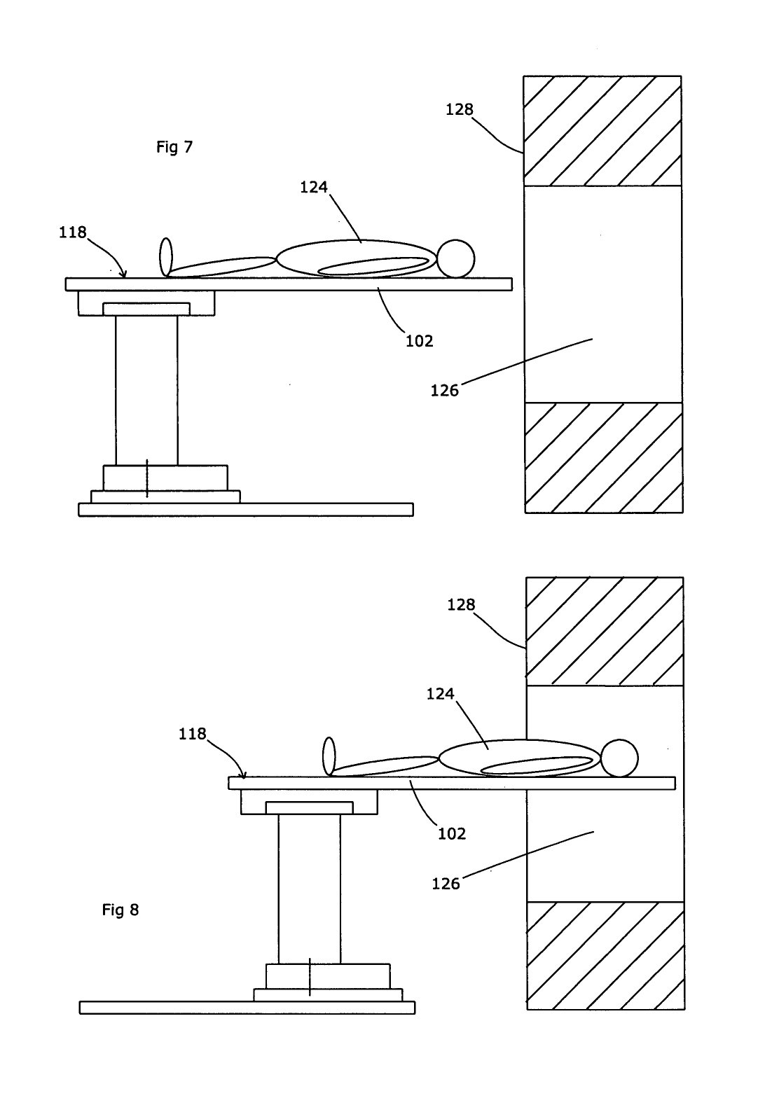

FIG. 7 shows the patient support 118 of FIG. 6 in the set-up position prior to treatment. A patient 124 is positioned on the table-top 102, at which point their position is determined accurately. If necessary, this position can be adjusted by way of the two translational motors 108, 110.

Once the responsible clinician is content with the position of the patient 124 on the support 118, the translational base drive motor 120 is activated to move the patient support and place the patient 124 into the aperture 126 of the radiotherapy apparatus 128. In doing so, the movement of the support 118 on the rails (or other means) leaves the geometry of the table top 102 and its mechanical support unchanged. Movement is generally in the lower sections of the support only, laving the upper sections unaffected. As a result, vertical deflection of the treatment site as a result of longitudinal movement is minimised.

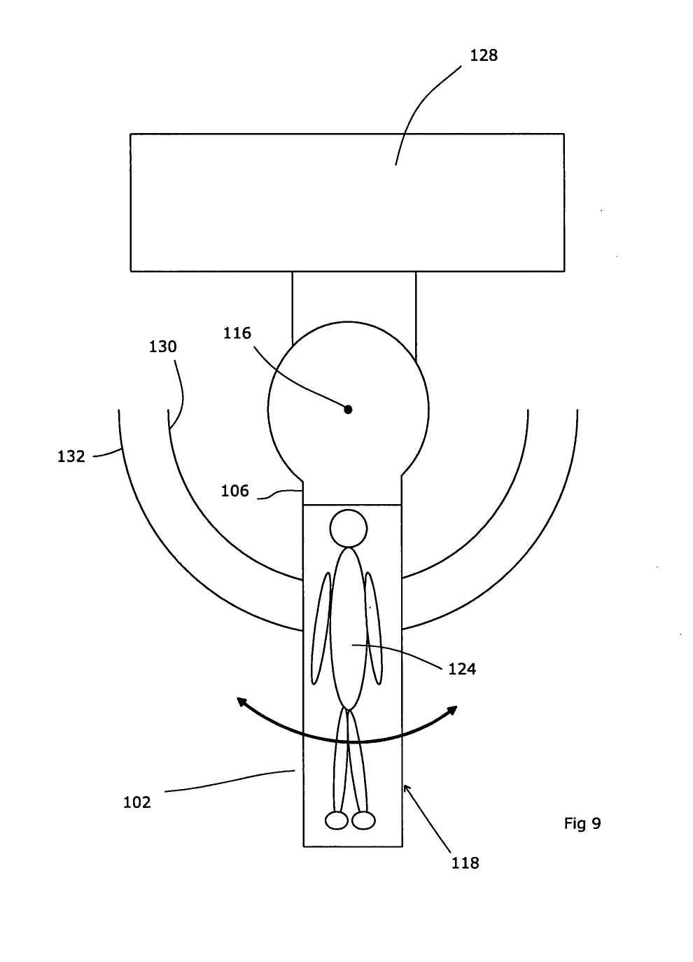

FIG. 9 shows the patient support from above. To allow for the absence of the base drive motor for rotation of the support about the vertical isocentre axis 116, the base unit 106 is mounted on circular concentric rails 130, 132 centred on the isocentre axis 116. This avoids the apparent impossibility of journaling the base unit 106 at the isocentre axis 116, since when in the treatment position the isocentre will be within the treatment apparatus. A motor beneath the table allows the base unit 106 to be rotated around the isocentre axis 116 by travelling along the rails 130, 132.

Thus, the present invention provides a patient support system that retains all the functionality of existing systems but which, in addition, does not suffer from a variable droop of the table top as the treatment region is placed into the treatment location. Improvements in the accuracy of patient positioning derived through this invention allow for greater refinement of the treatment given and hence allows for improvement in patient outcome.

It will of course be understood that many variations may be made to the above-described embodiment without departing from the scope of the present invention.

1. A patient support system, comprising a base, an upstanding support, and a couch attached to the support and having a cantilever section extendable beyond the support;
the support including a couch drive means to adjust the position of the couch relative to the support;
further comprising a support drive means for translationally driving the support relative to the base in the direction of the cantilever section.

2. A patient support system according to claim 1 in which the support drive means is located in the base.

3. A patient support system according to claim 1 in which the support drive means is located in the support.

4. A patient support system according to claim 1 in which the support drive means includes two fixed end stops between which the support is moved.

5. A patient support system according to claim 1 in which the support drive means is further adapted to drive the support in rotation about a vertical axis.

6. A patient support system according to claim 1 in which the couch drive means is adapted to adjust the height the couch relative to the base.

7. A patient support system according to claim 1 in which the couch drive means is adapted to displace the couch in the direction of the cantilever section.

8. A patient support system according to claim 1 in which the couch drive means is adapted to displace the couch in a direction transverse to the direction of the cantilever section.

9. A patient support system according to claim 1 in which the couch drive means is adapted to rotate the couch about a vertical axis.

10. A patient support system according to claim 1 in which the couch drive means is adapted to rotate the couch about one or more horizontal axes.

11. A patient support system according to claim 1 in which the base is a section of floor.

12. A patient support system according to claim 1 in which the base is a fixing for attachment to a floor.

13. Radiotherapy apparatus, comprising;
a patient support having a base, an upstanding support, and a couch attached to the support, the couch having a cantilever section extendable beyond the support, the support including a couch drive means to adjust the position of the couch relative to the support and a support drive means for translationally driving the support relative to the base in the direction of the cantilever section;
an enclosed treatment area positioned in line with the cantilever section.

14. Radiotherapy apparatus according to claim 13 further comprising optical position detection apparatus for a patient on the couch.

15. Radiotherapy apparatus according to claim 14 in which the optical position detection apparatus comprises a laser alignment system.

16. Radiotherapy apparatus according to claim 14 in which the optical position detection apparatus comprises a video camera.

17. Radiotherapy apparatus according to claim 14 in which the patient is not visible to the optical position detection apparatus when the cantilever section is extended into the enclosed treatment area.
